(19) United States
(12) Patent Application Publication
(10) Pub. No.: US 2025/0339173 A1
(43) Pub. Date: Nov. 6, 2025

(54) NEONATE LUMBAR PUNCTURE DEVICE

(71) Applicant: Ann and Robert H. Lurie Children's Hospital of Chicago, Chicago, IL (US)

(72) Inventor: Shankar Rajeswaran, Chicago, IL (US)

(21) Appl. No.: 18/854,435

(22) PCT Filed: Apr. 4, 2023
(86) PCT No.: PCT/US2023/065319

§ 371 Date: Oct. 4, 2024

Related U.S. Application Data
(60) Provisional application No. 63/327,338, filed on Apr. 4, 2022.

(51) Int. Cl. A61B 17/34

(52) U.S. Cl. CPC A61B 17/3401; A61B 17/3403; A61B 2017/3407; A61B 2017/3411; A61B 2017/3413

(57) ABSTRACT

One medical device may include an alignment portion configured to align the medical device with a spine of a patient. the alignment portion including a first end and a second end. The medical device may also include an insertion portion extending outward from the alignment portion and coupled to the alignment portion at the first end of the alignment portion. where the alignment portion and the insertion portion form an insertion angle. and where the insertion portion includes a set of needle insertion tubes, wherein each needle insertion tube is configured to receive a needle and guide the needle into the patient based on the insertion angle.

### REFERENCE TO RELATED APPLICATIONS

This application claims priority to U.S. Provisional Application No. 63/327,338, filed on Apr. 4, 2022, the entire contents of which is incorporated herein by reference.

### FIELD

The embodiments described herein relate to a medical device for performing neonate lumbar punctures, and, more particularly, to a neonate lumbar puncture device that increases a success rate of neonatal lumbar punctures performed by first-line medical provider.

### BACKGROUND

Neonatal lumbar punctures are routine procedures performed by medical providers and personnel, such as, for example, emergency room physicians and pediatricians. Currently, the procedure is performed blindly using landmarks on the neonate. In some cases, the medical provider is unsuccessful at obtaining cerebrospinal fluid (“CSF”). Such cases are subsequently referred to pediatric interventional radiology (“IR”). The IR physician is able to perform the neonatal lumbar puncture using ultrasound guidance.

The thecal sac, which contains CSF, is protected by the spine. There are small needle windows to access the thecal sac, which allows medical providers to perform lumbar punctures. As an adult, the window to a lumbar puncture is highly variable based on degenerative changes of the spine and the patient's habitus. This high level of variability does not exist in neonates. Rather, there is limited variation as to the insertion angle used to access the thecal sac in neonates, which is why the angle of approach (for example, the insertion angle) and depth (from skin surface to the CSF space) in the lower spine is reproducible in neonates.

### SUMMARY OF THE DISCLOSURE

Accordingly, embodiments described herein provide a medical device that aids medical providers (such as, for example, front-line medical providers) in performing neonatal lumbar punctures. For example, embodiments described herein provide a needle guide device. The needle guide device may be positioned along a patient's lower spine and provide an insertion angle that allows a needle to pass into the CSF space. Alternatively, or in addition, in some embodiments, the needle guide device may prevent the needle from being advanced too far into the patient. By controlling the insertion angle and degree of penetration or insertion of the needle, the embodiments described herein may increase a medical provider's success rate when performing neonatal lumbar puncture, which ultimately improves patient care and experience. Additionally, the embodiments described herein may minimize the need to escalate or refer a patient (or case) to pediatric IR, which expedites testing results, reduces costs, and further improves patient care and experience.

Accordingly, embodiments described herein provide a medical device for performing neonate lumbar punctures and systems and methods for performing neonate lumbar puncture procedures using the medical device. For example, one embodiment provides a medical device for performing neonate lumbar punctures. The medical device includes an alignment portion configured to align the medical device with a spine of the patient. The alignment portion including a first end and a second end. The medical device also includes an insertion portion extending outward from the alignment portion and coupled to the alignment portion at the first end of the alignment portion, wherein the alignment portion and the insertion portion form an insertion angle, and wherein the insertion portion includes a set of needle insertion tubes, wherein each needle insertion tube is configured to receive a needle and guide the needle into the patient based on the insertion angle.

Some embodiments described herein provide a method for performing neonatal lumbar punctures. The method includes providing a medical device. The medical device includes an alignment portion configured to align the medical device with a spine of a patient, the alignment portion including a first end and a second end, and an insertion portion extending outward from the alignment portion and coupled to the alignment portion at the first end of the alignment portion, wherein the alignment portion and the insertion portion form an insertion angle. The method also includes aligning, via the alignment portion, the medical device with the spine of the patient. The method also includes receiving, via the insertion portion, a needle through at least one needle insertion tube included in a set of needle insertion tubes of the insertion portion. The method also includes guiding, via the at least one needle insertion tube, into the patient based on the insertion angle.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1A illustrates a patient in a lateral position with the patient's spine flexed.

FIG. 1B illustrates a needle passing into the CSF space between vertebrae.

FIGS. 2A-2C illustrate a medical device for performing neonatal lumbar punctures according to some embodiments.

FIGS. 3A-3C illustrate an insertion portion of a medical device for performing neonatal lumbar punctures according to some embodiments.

FIGS. 4A-4B illustrate an alignment of the medical device of FIGS. 2A-2C along a patient's spine according to some embodiments.

FIG. 5 illustrates a medical device including a first tab and a second tab according to some configurations.

### DETAILED DESCRIPTION

A neonatal lumbar puncture is a medical procedure where a needle is inserted into the spinal canal to, among other things, collect cerebrospinal fluid (“CSF”) for diagnostic testing. In the field of pediatrics, a neonatal lumbar puncture is a common procedure for analyzing CSF to diagnose, for example, meningitis, sepsis, fever, subarachnoid hemorrhage, and the like. Accordingly, neonatal lumbar punctures are routine procedures performed by medical provider, such as, for example, front-line emergency room physicians and pediatricians.

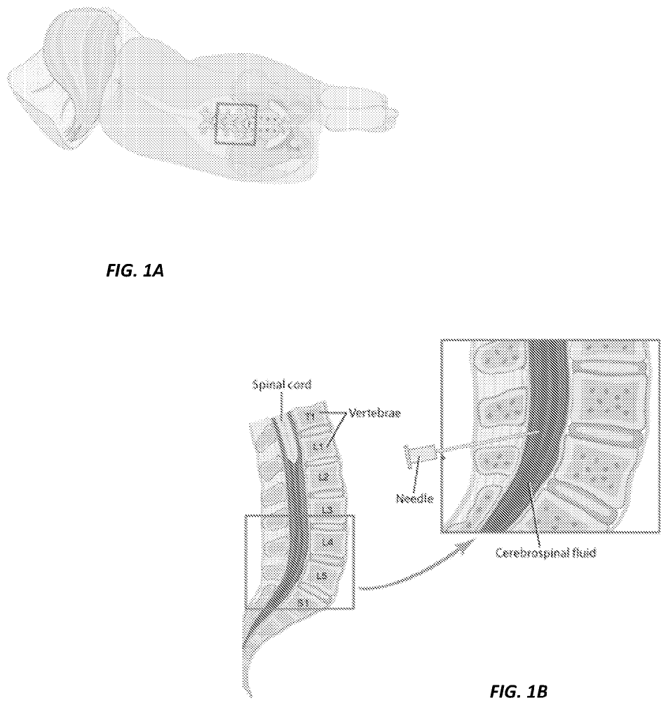

Conventionally, the procedure is performed blindly using landmarks on the neonate. When performing the procedure, the medical provider positions the patient in a sitting or lateral position (for example, a laying position) with the patient's spine flexed. As one example, FIG. 1A illustrates a patient in a lateral position with the patient's spine flexed. Once the patient is properly positioned, the medical provider cleans the insertion site with a sterile antiseptic solution, such as, for example, ChloraPrep. After the insertion site is dry, the medical provider may place a sterile towel under the patient. Additionally, the medical provider may drape the patient with sterile towels or place a sterile 4×4 on the iliac crest of the patient.

At the level of the iliac crest, the medical provider palpates the third intervertebral space (between the L3-L4 vertebrae or L4-L5 vertebrae). As illustrated in FIG. 1B, the medical provider inserts a needle (for example, a #22 gauge spinal needle or a #23 gauge spinal needle) slowly with a stylet in place into the intervertebral space toward the umbilicus. The medical provider may use two fingers to guide the needle and thumbs to slowly advance. The medical provider may advance the needle several millimeters at a time and withdraw the stylet frequently to check for CSF flow. In small patients, the medical provider may not feel a “pop” when the dura is penetrated. When the medical provider feels resistance, the medical provider may withdraw the needle to the skin surface and redirect the angle slightly (for example, as an unsuccessful first attempt). After redirecting the insertion angle, the medical provider repeats the steps outlined above (as subsequent attempts). Once the medical provider observes CSF flow (as a successful attempt), the medical provider may allow the CSF to drip by gravity. The medical provider collects the CSF (for example, 0.5 to 1 mL per tube), reinserts the stylet, and removes the needle. After collecting the desired amount of CSF and removing the needle, the medical provider may apply pressure to the insertion site, clean the sterile antiseptic solution from the patient's skin, and place a bandage over the insertion site.

As noted above, in some instances, the medical provider performing the procedure is unsuccessful at obtaining CSF. As one example, the medical provider may not be successful on a first attempt but may be successful on a subsequent attempt (for example, after removing the needle and adjusting an insertion angle of the needle). As another example, the medical provider may not be successful after a number of attempts (for example, after removing the needle and adjusting an insertion angle of the needle multiple times). In such instances, the patient is referred to pediatric interventional radiology (“IR”), where an IR physician performs the neonatal lumbar puncture using ultrasound (“US”) guidance.

The thecal sac, which contains CSF, is protected by the spine. There are small needle windows to access the thecal sac, which allows medical providers to perform lumbar punctures (as illustrated in FIG. 1B). As an adult, the window to a lumbar puncture is highly variable based on degenerative changes of the spine and the patient's habitus. This high level of variability does not exist in neonates. Rather, there is limited variation as to the insertion angle used to access the thecal sac in neonates, which is why the angle of approach (for example, the insertion angle) and depth (for example, the skin surface to the thecal sac) is reproducible in neonates. By controlling the depth a needle may be inserted, a needle may be prevented from being inserted too deep and adjacent to critical structures, which avoids patient harm. Accordingly, embodiments described herein provide a medical device that aids medical provider (such as, for example, front-line medical providers) in performing neonatal lumbar punctures.

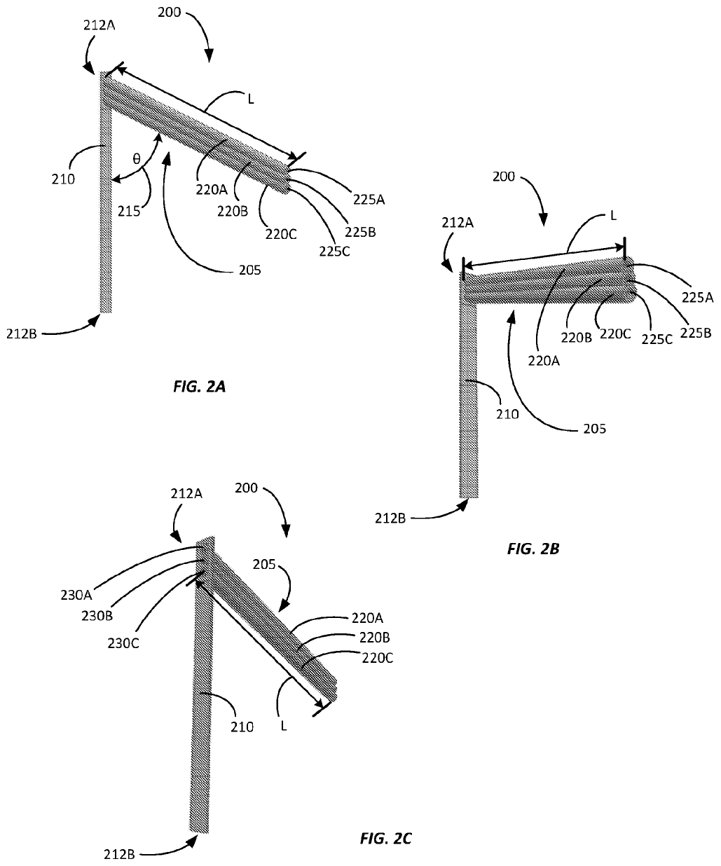

FIGS. 2A-2C illustrate a medical device 200 for performing neonatal lumbar punctures according to some embodiments. The medical device 200 may be formed (or made) using plastic (or another suitable polymer), metal, another acceptable material, or a combination thereof. As one example, the medical device 200 may include a metal lining (e.g., lining an interior of a needle insertion tube, as described in greater detail below) to prevent debris (e.g., plastic debris) being created by contact with the needle and potentially contaminating the patient upon needle insertion.

As illustrated in FIGS. 2A-2C, the medical device 200 may include an insertion portion 205 and an alignment portion 210. In the illustrated example, the insertion portion 205 is coupled to the alignment portion 210. In some embodiments, the insertion portion 205 is permanently coupled to the alignment portion 210. Alternatively, in some embodiments, the insertion portion 205 is temporarily coupled to the alignment portion 210, such that the insertion portion 205 and the alignment portion 210 may be decoupled or detached. In other embodiments, the medical device 200 does not include the alignment portion 210.

As also illustrated in FIGS. 2A-2C, when the insertion portion 205 and the alignment portion 210 are coupled, the insertion portion 205 and the alignment portion 210 form an insertion angle 215. The insertion angle 215 represents or corresponds to an angle that allows a needle to pass into the CSF space (as illustrated in FIG. 1B). As noted above, the insertion angle 215 may be consistent (or nearly consistent) across neonates. Accordingly, in some embodiments, the insertion angle 215 is a fixed angle, such as a fixed angle that is consistent (or nearly consistent) across neonates. In some embodiments, the insertion angle 215 is a fixed angle between approximately 60 degrees and 70 degrees. Alternatively, in some embodiments, the insertion angle 215 may be a dynamic angle (e.g., an adjustable angle). For example, in such embodiments, the insertion angle 215 may be based on one or more patient-specific characteristics or measurements (e.g., a specific population, medical condition, patient age, patient situation, or the like). As one example, a medical provide may adjust the insertion angle 215 to take into account patient-specific characteristics or measurements (e.g., where the insertion angle is dependent on the patient-specific characteristics or measurements). Accordingly, in some embodiments, the insertion angle 215 may be associated with patient-specific characteristics or measurements.

In some embodiments, the medical device 200 is included in a medical kit. In such embodiments, the medical kit may include a set of medical devices 200 (for example, a first medical device, a second medical device, a third medical device, and the like). Each medical device included in the set of medical devices 200 may include one or more different parameters. In some embodiments, each medical device included in the set of medical devices 200 may use (or implement) a different insertion angle. As one example, a first medical device may use an insertion angle of 60 degrees, a second medical device may use an insertion angle of 65 degrees, and a third medical device may use an insertion angle of 70 degrees. Alternatively, or in addition, in some embodiments, each medical device included in the set of medical devices 200 may be associated with a different needle characteristic, such as a needle length, a needle gauge, or the like. As one example, a first medical device may be associated with a first needle length and a second medical device may be associated with a second needle length different from the first needle length. Alternatively, or in addition, in some embodiments, each medical device included in the set of medical devices 200 may be associated with a different patient characteristic or parameter, such as a weight. As one example, a first medical device may be associated with a first patient weight and a second medic al device may be associated with a second patient weight different than the first patient weight. Alternatively, or in addition, in some embodiments, each medical device included in the set of medical devices 200 may be associated with a different patient position (e.g., a position of the patient during performance of the procedure). As one example, a first medical device may be associated with a lateral position of the patient during the procedure and a second medical device may be associated with a sitting position of the patient during the procedure.

The alignment portion 210 is configured to align or position the medical device 200 (for example, the insertion portion 205) along the patient's spine such that when a needle is inserted via the insertion portion 205 (as described in greater detail below), the needle may pass into the CSF space. As illustrated in FIGS. 2A-2C, the alignment portion 210 includes a first end 212A and a second end 212B. The alignment portion 210 is coupled to the insertion potion 205 at (or near) the first end 212A. As illustrated in FIG. 2C, the first end 212A is configured to allow the needle (received via the insertion portion 205) to pass through the alignment portion 210 and, ultimately, pass into (or penetrate) the patient. In some embodiments, the first end 212A includes a series of voids or openings, where each void or opening corresponds with a needle insertion tube (as described in greater detail below). A distance between voids may be such that if a first attempt is unsuccessful (e.g., meets bone), a second or third attempt may increase a likelihood of reaching the thecal sac (when the medical device 200 remains in a fixed position), where the second or third attempt is associated with a different void than the first attempt. In some embodiments, the second end 212B is configured to be aligned in a cranial-caudal direction with the gluteal cleft, the iliac crest, or the like of the patient. For example, when positioning the medical device 200 along the patient's spine, the medical provider may first align the second end 212B at the gluteal cleft of the patient and then position the first end 212A along the spine such that the alignment portion 210 runs parallel with the patient's spine. Accordingly, the alignment portion 210 may function as an extension to the gluteal cleft for a landmark of midline and cranial-caudal offset to L3-L5, which is consistent (or near consistent) across neonates. By using the gluteal cleft as a single landmark, a medical provider may establish both midline and a fixed cranial-caudal location.

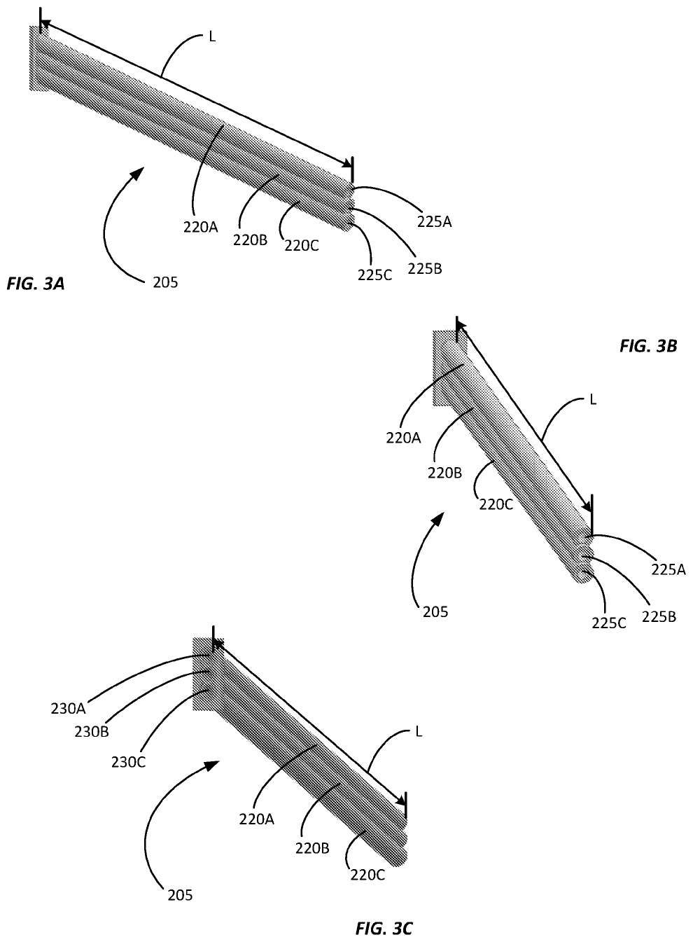

In some embodiments, the medical device 200 includes an integrated adhesive to secure the medical device 200 to a patient's back during a procedure. In such embodiments, the adhesive may be positioned on the back of plastic wings extending out on both sides of the needle holes (for example, the outlets 230 shown as a set of voids or openings illustrated in FIG. 2C). Alternatively, or in addition, in some embodiments, an adhesive is positioned directly around a needle hole, in an extension (e.g., a plastic extension) positioned around a needle hole (e.g., positioned in any direction around the needle hole), or a combination thereof. The adhesive improves puncturing because, once placed, the adhesive (for example, the medical device 200) will stay in the proper anatomical positioning (for example, even when the patient moves during the procedure). In some configurations, the adhesive may enable a medical provider to remove a hand from the medical device 200 such that the medical provider may utilize the removed hand (or both hands) to collect a sample without the medical device 200 moving away from the proper anatomical positioning. The medical device 200 overall assists in accurate placement because the medical device 200 utilizes landmarks in the cranial-caudal and medial-lateral directions, as well as the fixed angle of insertion, which may be held constant by the adhesive. Alternatively, or in addition, in some embodiments, the integrated adhesive is positioned along the entire surface of the alignment portion 210 that comes in contact with the patient's back. Alternatively, or in addition, the integrated adhesive may be positioned along a sub-portion of the surface of the alignment portion 210 that comes into contact with the patient's back (for example, the set of voids or openings illustrated in FIG. 2C). Alternatively, or in addition, in some embodiments, the medical device 200 may incorporate a belt or strap for securement. In such embodiments, the belt or strap may be fastened or temporarily coupled to the patient. Alternatively, or in addition, in some embodiments, where there is no downward extension (e.g., as illustrated in FIGS. 3A-3C), the medical device 200 may include one or more extensions (e.g., short plastic extensions). The one or more extensions may be in the left-right direction, the cranial-caudal direction, or a combination thereof to assist in positioning the medical device 200. As one example, a medical provider may use their non-dominant hand to hold the medical device 200 against the patient's back via the one or more extensions while using their dominant hand to manipulate the needle.

As illustrated in FIGS. 2A-2C, the insertion portion 205 includes a set of needle insertion tubes 220. In the illustrated example, the insertion portion 205 includes a first needle insertion tube 220A, a second needle insertion tube 220B, and a third needle insertion tube 220C. Although the insertion portion 205 is illustrated with three needle insertion tubes 220, it should be understood that the insertion portion 205 may include additional or fewer needle insertion tubes 220. As one example, the insertion portion 205 may include a single needle insertion tube 220 (for example, the first needle insertion tube 220A). As another example, the insertion portion 205 may include more than three needle insertion tubes 220 (for example, the first needle insertion tube 220A, the second needle insertion tube 220B, the third needle insertion tube 220C, and so on upwards of “N” total needle insertion tubes).

A needle insertion tube 220 is configured to receive and guide a needle (such as a needle used to perform a neonatal lumbar puncture). For example, a needle insertion tube 220 may ensure that a needle is inserted into the patient at a specified position, depth, angle, or a combination thereof. In other words, the needle insertion tube 220 controls (or standardizes) how a needle is inserted into a patient based on, for example, how the medical device 200 is positioned or aligned with a patient (via, for example, the alignment portion 210). For example, the needle insertion tube 220 ensures that a needle is inserted at an insertion angle that allows a needle to pass into the CSF space (for example, the insertion angle 215). Alternatively, or in addition, the needle insertion tube 220 may prevent a needle from being advanced too far into a patient, not far enough into a patient, or a combination thereof (for example, may control or standardize an insertion or penetration depth).

The set of needle insertion tubes 220 are positioned or arranged such that each needle insertion tube 220 is positioned adjacent to each other in the cranial-caudal direction. In other words, each of the needle insertion tubes 220 are positioned or arranged along the insertion portion 205 such that each of the needle insertion tubes 220 have a cranial-caudal offset. For example, the first needle insertion tube 220A is adjacent (or proximate) to the second needle insertion tube 220B by a cranial-caudal offset and the second needle insertion tube 220B is adjacent (or proximate) to the third needle insertion tube 220C by a cranial-caudal offset. Accordingly, each needle insertion tube 220 is associated with a different cranial-caudal position or placement (for example, along a patient's spine). Although the cranial-caudal offset from the gluteal cleft is consistent across neonates, it may vary slightly if the neonate's (patient's) back is straight versus curved. Accordingly, by including more than one needle insertion tube 220, where each needle insertion tube 220 is associated with a different position or placement along the patient's spine, a medical provider may experience a higher likelihood of success on subsequent attempts after an unsuccessful first attempt (for example, by providing fixed offsets from previous attempts). Accordingly, each needle insertion tube 220 is offset from each other to account for variability in placement caused by movement, anatomy variations, or the like. In some embodiments, this offset (e.g., distance) is determined based on an average distance between vertebra in neonates. For example, offsets account for a distance between vertebra such that if a first attempt hits a vertebra the second or third attempt will be offset appropriately and, therefore, may be less likely to hit the same vertebra (but the offset is not big enough to reach an adjacent vertebra).

Accordingly, by including more than one needle insertion tube 220 (as illustrated in FIGS. 2A-2C), the medical provider may perform subsequent attempts with a cranial-caudal offset (for example, using a different needle insertion tube 220) without having to readjust the medical device 200. As one example, on a first attempt, the medical provider may perform the neonatal lumbar puncture using the first needle insertion tube 220A. However, the medical provider may experience resistance when inserting the needle through the first needle insertion tube 220A. In response to experiencing resistance (for example, an unsuccessful first attempt), the medical provider may remove the needle and re-insert the needle into the second needle insertion tube 220B (as a second attempt). When the medical provider experiences resistance on the second attempt (for example, an unsuccessful second attempt), the medical provider may remove the needle and re-insert the needle into the third needle insertion tube 220C (as a third attempt). Accordingly, by utilizing different needle insertion tubes 220 on different attempts, the medical provider is, ultimately, adjusting an insertion point or position along the patient's spine.

As illustrated in FIGS. 2A-2C, each needle insertion tube 220 includes a corresponding inlet 225 and a corresponding outlet 230. For example, a first inlet 225A and a first outlet 230A correspond with the first needle insertion tube 220A, a second inlet 225B and a second outlet 230B correspond with the second needle insertion tube 220B, and a third inlet 225C and a third outlet 230C correspond with the third needle insertion tube 220C. An inlet 225 is configured to receive a needle (for example, as an entrance point for a needle being received by a needle insertion tube 220). An outlet 230 is configured to output the needle received by the corresponding inlet 225 (for example, as an exit point for the needle being received by the needle insertion tube 220). As one example, the first inlet 225A may receive a needle. After the needle is received by the first inlet 225A, the needle traverses (or is guided) along the length L of the first needle insertion tube 220A. The needle then exits the first needle insertion tube 220A via the first outlet 230A and penetrates the patient.

Each needle insertion tube 220 has a length L. The length L of a needle insertion tube 220 may be (or represent) a distance between the corresponding inlet 225 and the corresponding outlet 230 of the needle insertion tube 220. The length L of a needle insertion tube 220 may represent or correspond to an insertion (or penetration) depth of the needle. For example, the length L of a needle insertion tube 220 may define how deep a needle will penetrate a patient. An insertion depth is consistent (or nearly consistent) across neonates. Accordingly, the needle insertion tube 220 (via a length L of the needle insertion tube 220) may standardize an insertion depth. A standardized (or controlled) insertion depth may increase a medical provider's first-attempt success rate in performing neonatal lumbar punctures. As one example, when using a 1.5 inch needle, the length L may be defined such that the 1.5 inch needle cannot extend into the patient beyond a safe distance (e.g., approximately 2 cm for a neonate). The diameter of the needle insertion holes 220 may prevent the proximal end of the needle (e.g., the needle end furthest from the patient) from entering through the insertion hole (e.g., only the needle, not the plastic applicator at the proximal end of the needle, will fit through the needle insertion tube 220).

In some embodiments, each needle insertion tube 220 has the same length L, as illustrated in FIGS. 2A-2C. Alternatively, or in addition, in some embodiments, two or more needle insertion tubes 220 can have different lengths (not illustrated), such that, e.g., a medical provider may utilize different needle lengths while still achieving the same permissible depth of insertion. For example, the first needle insertion tube 220A may have a first length, the second needle insertion tube 220B may have a second length, and the third needle insertion tube 220B may have a third length. Following this example, in some embodiments, the first length and the second length may be the same while the third length is different from the first length and the second length. As another example, the first length, the second length, and the third length may each be different. Accordingly, in some embodiments, a subset of the set of needle insertion tubes 220 may have the same length. Alternatively, or in addition, in some embodiments, a subset of the set of needle insertion tubes 220 may have different lengths.

In some embodiments, the length L of the needle insertion tube 220 is fixed. However, in other embodiments, the length L of the needle insertion tube 220 is adjustable. For example, in some embodiments, the needle insertion tube 220 may be adjustable or telescoping. Alternatively, or in addition, the needle insertion tube 220 may be configured to receive a tube extension component. The tube extension component may extend the length L of the needle insertion tube 220 such that the total length of the needle insertion tube 220 when the tube extension component is coupled to the needle insertion tube 220 results in a specific penetration or insertion depth corresponding to a specific needle length being used. The tube extension component may be removably coupled or fastened to the needle insertion tube 220. As one example, the tube extension component may be magnetically coupled to the needle insertion tube 220. As another example, the tube extension component may be a clip-on component that clips onto the needle insertion tube 220. As yet another example, the tube extension component may be an adhesive component that adheres onto the needle insertion tube 220.

The length L may be adjusted to a desired or recommended length based on a characteristic or measurement associated with the patient, such as a weight, a length, a gestational age of the patient at the patient's birth, whether the patient was a full-term neonate or a pre-term neonate, and the like. As one example, when the patient was greater than 37 weeks at birth, the recommended (or optimal) insertion depth may be approximately 11 mm. According to this example, the medical provider may adjust the length L of the needle insertion tube 220 such that the penetration depth is approximately 11 mm. As another example, when the patient was less than 37 weeks at birth, the recommended insertion depth may be approximately 14 mm. According to this example, the medical provider may adjust the length L of the needle insertion tube 220 such that the penetration depth is approximately 14 mm. Accordingly, a medical provider may adjust the needle insertion tube 220 (for example, to a preset length) based on a characteristic or measurement associated with the patient, a characteristic or measurement associated with the needle (e.g., needle length), or a combination thereof. Accordingly, in some embodiments, the length L of the needle insertion tube 220 may be dynamically adjusted by the medical provider at various increments (for example, along an incremental scale). As one example, the medical provider may adjust the length L of the needle insertion tube 220 at increments of 1 mm.

Alternatively, or in addition, the length L of the needle insertion tube 220 may be set based on a formula, such as, for example, 2.5×patient weight (kg)+6 (mm). Accordingly, in some embodiments, the medical provider may adjust the length L of the needle insertion tube 220 along an incremental scale based on an outcome of the formula noted above.

Alternatively, or in addition, in some embodiments, the length L of the needle insertion tube 220 is set based on a needle characteristic, such as a length of a needle (needle length). As one example, when the medical provider is planning to use a 1 inch needle, the medical provider may adjust the length L of the needle insertion tube 220 to account for the 1 inch needle. As another example, when the medical provider is planning to use a 1.5 inch needle, the medical provider may adjust the length L of the needle insertion tube 220 to account for the 1.5 inch needle.

As noted above, in some embodiments, the medical device 200 may be included in a medical kit. The medical kit may include a set of medical devices 200 (for example, a first medical device, a second medical device, a third medical device, and the like), where each medical device 200 includes one or more different parameters. In some embodiments, each medical device 200 may use (or implement) a different length L for the needle insertion tubes 220. As one example, a first medical device may use a first length L for the needle insertion tubes 220, a second medical device may use a second length L for the needle insertion tubes 220, and a third medical device may use a third length L for the needle insertion tubes 220. Accordingly, in such embodiments, the medical provider may select the appropriate medical device 200 (based on a desired length L of the needle insertion tubes 220). As one example, a first medical device 200 may have a first length for the needle insertion tubes 220, where the first length L is associated with pre-term neonates, and a second medical device 200 may have a second length L for the needle insertion tubes 220, where the second length L is associated with full-term neonates. Following this example, when the patient is a full-term neonate, the medical provider may select the second medical device 200 from the medical kit. However, when the patient is a pre-term neonate, the medical provider may select the first medical device 200 from the medical kit. As another example, the medical kit may include a first medical device 200 configured to be used with a first needle length and a second medical device 200 configured to be used with a second needle length different from the first needle length. According to this example, the medical provider may select either the first medical device 200 or the second medical device 200 based on a length of needle, such as a length of needle that is available to the medical provider, a preferred length of needle for the medical provider, or the like. Alternatively, or in addition, in some embodiments, each medical device included in the set of medical devices 200 may be associated with a different combination of characteristics or measurements associated with a patient, a needle, or the like. As one example, a first medical device 200 may be associated with a 1 inch needle and a <37 week gestation patient and a second medical device 200 may be associated with a 1.5 inch needle and a <35 week gestation patient. Alternatively, or in addition, in some embodiments, the medical kit may include the medical device 200 and a needle of a specific length corresponding to the medical device 200. In such embodiments, the medical provider may perform the neonatal lumbar puncture with the medical device 200 using the needle included in the medical kit.

Alternatively, or in addition, in some embodiments, the medical device 200 only includes the insertion portion 205, as illustrated in FIGS. 3A-3C. In such embodiments, the insertion portion 205 functions as similarly described herein with respect to FIGS. 2A-2C. However, in such embodiments, a medical provider positions or aligns the insertion portion 205 (as the medical device 200) manually (for example, without aid of the alignment portion 210). As one example, a medical provider may palpate the patient to identify a probable insertion site along the patient's spine. After identifying the probable insertion site, the medical provider may place or align the insertion portion 205 (as the medical device 200) (based on the probable insertion site) such that the insertion portion 205 (for example, the needle insertion tube(s) 220) align with the probable insertion site. When the medical provider's first attempt is unsuccessful, the medical provider may adjust the actual insertion point by utilizing a different needle insertion tube 220 without, for example, moving or adjusting the placement of the insertion portion 205 (as the medical device 200) along the patient's spine.

In some embodiments, the medical device 200 is a clip-on lumbar puncture device that secures to other medical devices or equipment. For example, the medical device 200 may attach to a bedside ultrasound wand. In such embodiments, the medical device 200 may include a c-shaped extension (near a top portion of the medical device 200) that attaches (for example, snaps or clamps with a screw adjustment) to an ultrasound probe or wand (standard size). Such embodiments enable simultaneous (or near simultaneous) ultrasound use with the medical device 200 such that first-attempt success may be improved.

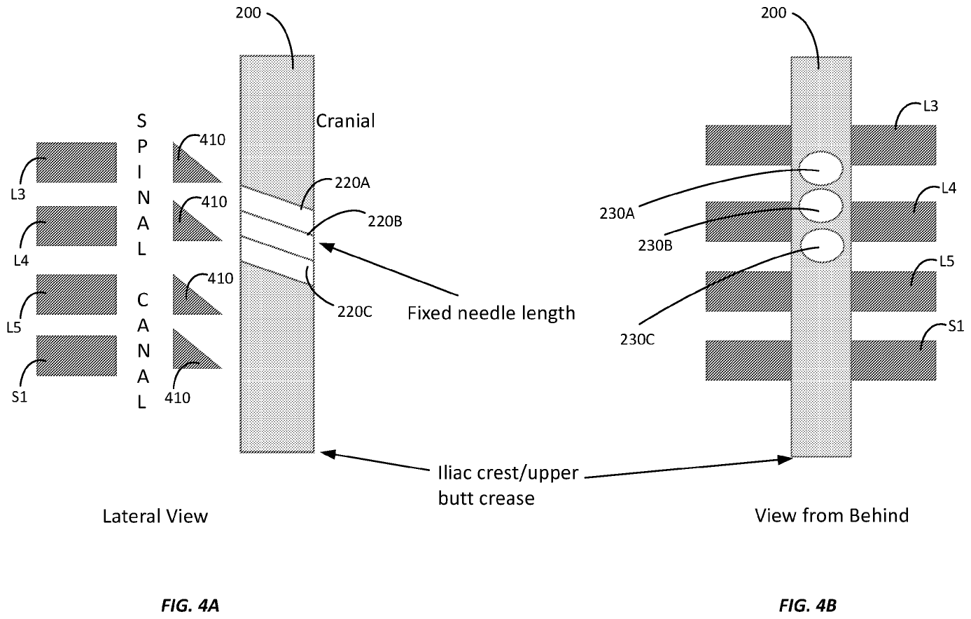

Accordingly, in some embodiments, the medical device 200 uses the iliac crests or the gluteal cleft as the inferior margin. Alternatively, or in addition, in some embodiments, the medical device 200 uses a fixed distance (cranial) to mark the L3-4 or L4-5 vertebrae level. For example, as illustrated in FIGS. 4A-4B, the cranial aspect is lined with the spinous process (represented in FIGS. 4A-4B by reference numerals 410) so there is no lateral deviation. As also illustrated in FIGS. 4A-4B, multiple side holes (for example, the set of outlets 230) are angled in a direction (as illustrated in FIG. 4A) that is parallel with the inferior edge of the spinous process 410. As noted above, the medical device 200 does not allow the needle to advance beyond a fixed depth (for example, the insertion or penetration depth).

In some configurations, the medical device 200 may include one or more tabs. A tab may provide a medical provider an option for holding the medical device 200 in a more ergonomic manner, such as, for example, when the patient is placed in a lateral position. As one example, the medical provider may interact with the tab(s) (e.g., to hold the medical device 200) during insertion of the needle.

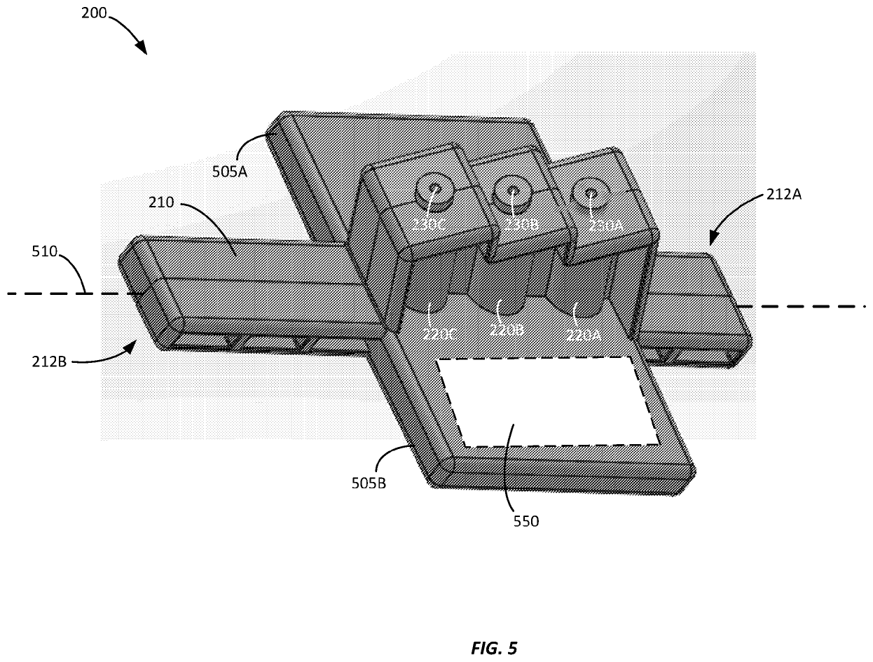

For example, FIG. 5 illustrates the medical device 200 including a first tab 505A and a second tab 505B according to some configurations. As illustrated in FIG. 5, the first and second tabs 505A, 505B may extend laterally outward from the alignment portion 210 (e.g., perpendicularly from a centerline represented in FIG. 5 by reference numeral 510). For instance, in some configurations, as illustrated in the example of FIG. 5, the first tab 505A may extend outward in a first direction and the second tab 505B may extend outward in a second direction opposite the first direction. While the first tab 505A and the second tab 505B are extending outward directly opposite each other from the same (or substantially similar) position in the example of FIG. 5, in some configurations, the first tab 505A and the second tab 505B may be offset such that the first tab 505A extends outward from a first position and the second tab 505B extends outward from a second position different from the first position.

Alternatively, or in addition, in some configurations, the medical device 200 may include additional, fewer, or different tabs in different configurations, sizes, or shapes than illustrated in FIG. 5. As one example, the medical device 200 may include a single tab (e.g., the first tab 505A), where the single tab is positioned based on a dominate or preferred hand of the medical device 200 (e.g., a left hand or right hand). As another example, the medical device 200 may include a third tab, where the third tab may be positioned on the same side of the medical device 200 as the first tab 505A, the same side of the medical device 200 as the second tab 505B, the first end 212A, the second end 212B, etc. Alternatively, or in addition, one or more of the tabs may extend outward from the medical device 200 at an alternative or different angle or direction than illustrated in FIG. 5 by the first tab 505A and the second tab 505B. In some configurations, one or more of the tabs may be triangular in shape. For instance, a tab may be triangular in shape such that a lateral portion of the tab is a wider portion of the triangle to, e.g., accommodate one or more fingers of a medical provider during positioning of the medical device 200. A medial portion of the triangular tab may be a narrower portion of the triangular tab, which may decrease a footprint of the medical device 200 closest to the needle insertion site such that visibility to the user is improved.

In configurations where the medical device 200 includes one or more tabs, an overall footprint (or square surface area) of the medical device 200 may be increased, which may, ultimately, result in decreased visibility of the application site or region from the perspective of the medical provider. Accordingly, in some configurations, the medical device 200 (or a portion thereof, such as, e.g., the tab(s)) may be formed of a translucent (or partially translucent) material or substance. In such configurations, the medical provider may be able to see the application site or region through an obstruction created by the medical device 200 (or portion thereof).

Alternatively, or in addition, in some configurations, the medical device 200 may include an imprinting element or component. An imprinting element may cause an imprint on (or otherwise mark) a patient at an application site or region. In some instances, the imprint is associated with an insertion site (e.g., where the needle will insert into the patient). The imprinting element may be a protrusion formed within the medical device 200. Accordingly, in some instances, when the medical device 200 is positioned on a patient, the medical device 200 (via the imprinting element or component) causes an imprint (or marking) on the patient such that when the medical device 200 is removed from the patient, the medical provider may see where the needle will insert into the patient by the imprint (or marking) left on the patient.

Alternatively, or in addition, in some configurations, the tab (e.g., the first tab 505A, the tab 505B, etc.) may include a void or opening. The void or opening may decrease the footprint of the medical device 200 (e.g., enable a medical provider to see more of the application site or region through the void or opening when positioning the medical device 200). For example, FIG. 5 illustrates an example void or opening with respect to the second tab 505B (represented in FIG. 5 by reference numeral 550).

Alternatively, or in addition, in some configurations, the tab (e.g., the first tab 505A, the tab 505B, etc.) may be adjustable (e.g., about a fixed axis). For instance, the tab may be attached (or otherwise coupled) to the medical device 200 (e.g., the alignment portion 210 or another portion thereof) by a rotation device (e.g., a hinge). The tab may be rotatable about the hinge such that the medical provider may reposition or adjust a position of the tab. For example, the medical provider may rotate (or fold) up the tab(s) when the medical device 200 is being placed on the patient, such that the medical provider has increased viewing of the application site or region. After the medical device 200 is positioned on the patient, the medical provider may then rotate (or fold) down the tab(s) such that, e.g., the medical provider may interact with the tab(s) while inserting a needle. In some configurations, the tab(s) may include an adhesive portion (e.g., such as a surface of the tab that contacts the patient). An adhesive portion may include an entire surface of the tab or a portion of the tab. Alternatively, or in addition, the tab may be removeable or detachable from the medical device 200 (e.g., the alignment portion 210 or another portion thereof).

The present disclosure has described one or more preferred embodiments, and it should be appreciated that many equivalents, alternatives, variations, and modifications, aside from those expressly stated, are possible and within the scope of the embodiments described herein.

What is claimed is:

1. A medical device for performing neonatal lumbar punctures, the medical device comprising:
an alignment portion configured to align the medical device with a spine of a patient, the alignment portion including a first end and a second end; and
an insertion portion extending outward from the alignment portion and coupled to the alignment portion at the first end of the alignment portion, wherein the alignment portion and the insertion portion form an insertion angle,
wherein the insertion portion includes a set of needle insertion tubes, wherein each needle insertion tube is configured to receive a needle and guide the needle into the patient based on the insertion angle.

2. The medical device of claim 1, wherein the insertion angle defines an angle at which the needle is inserted into the patient.

3. The medical device of claim 1, wherein each needle insertion tube included in the set of needle insertion tubes has a length.

4. The medical device of claim 3, wherein the length defines a penetration depth to which the needle is inserted into the patient.

5. The medical device of claim 3, wherein each needle insertion tube has the same length.

6. The medical device of claim 3, wherein the length is different for each needle insertion tube.

7. The medical device of claim 1, wherein each needle insertion tube is associated with a different cranial-caudal offset.

8. The medical device of claim 1, wherein the second end is configured to align with a gluteal cleft of the patient.

9. The medical device of claim 1, further comprising:
an integrated adhesive configured to secure the medical device to a back of the patient during a procedure.

10. The medical device of claim 1, further comprising:
an c-shaped extension configured to attach to another medical device.

11. The medical device of claim 10, wherein the other medical device is a component of an ultrasound machine.

12. A method for performing neonatal lumbar punctures, the method comprising:
providing a medical device, the medical device including
an alignment portion configured to align the medical device with a spine of a patient, the alignment portion including a first end and a second end, and
an insertion portion extending outward from the alignment portion and coupled to the alignment portion at the first end of the alignment portion, wherein the alignment portion and the insertion portion form an insertion angle;
aligning, via the alignment portion, the medical device with the spine of the patient;
receiving, via the insertion portion, a needle through at least one needle insertion tube included in a set of needle insertion tubes of the insertion portion; and
guiding, via the at least one needle insertion tube, into the patient based on the insertion angle.

13. The method of claim 12, wherein the insertion angle defines an angle at which the needle is inserted into the patient.

14. The method of claim 12, wherein each needle insertion tube included in the set of needle insertion tubes has a length.

15. The method of claim 14, wherein the length defines a penetration depth to which the needle is inserted into the patient.

16. The method of claim 14, wherein each needle insertion tube has the same length.

17. The method of claim 14, wherein the length is different for each needle insertion tube.

18. The method of claim 12, wherein each needle insertion tube is associated with a different cranial-caudal offset.

19. The medical device of claim 12, wherein aligning the medical device with the spine of the patient includes aligning the second end of the alignment portion with a gluteal cleft of the patient.

20. The method of claim 12, further comprising:
securing the medical device to a back of the patient during a procedure using an integrated adhesive of the medical device.

21. The method of claim 12, further comprising:
attaching the medical device to another medical device using a c-shaped extension of the medical device.

22. The method of claim 21, wherein attaching the medical device to another medical device includes attaching the medical device to a component of an ultrasound machine.
